(19) United States
(12) Patent Application Publication
(10) Pub. No.: US 2016/0256233 A1
(43) Pub. Date: Sep. 8, 2016

(54) TRAJECTORY GUIDE SYSTEMS, FRAMES AND METHODS FOR IMAGE-GUIDED SURGERIES

(71) Applicant: MRI Interventions, Inc., Irvine, CA (US)

(72) Inventors: Rajesh Pandey, Irvine, CA (US); Maxwell Jerad Daly, Redlands, CA (US)

(21) Appl. No.: 15/046,713

(22) Filed: Feb. 18, 2016

Related U.S. Application Data
(60) Provisional application No. 62/128,623, filed on Mar. 5, 2015.

(51) Int. Cl. A61B 90/11; A61B 5/055

(52) U.S. Cl. CPC A61B 90/11; A61B 5/055; A61B 2090/103

(57) ABSTRACT

A trajectory guide frame for use with an image-guided interventional system includes a base, an elongate device guide support, a device guide, and a locking device. The base has a patient access aperture formed therein. The elongate device guide support is secured to the base and has opposite proximal and distal ends. The distal end is positioned proximate the patient access aperture. The device guide support includes a device guide support bore therethrough that extends from the proximal end to the distal end. The device guide is configured to be removably inserted in the device guide support bore. The device guide includes a device guide lumen configured to removably receive an interventional device therethrough. The locking device is configured to secure the interventional device to the device guide and/or the device guide support. The locking device includes a gripping mechanism including: a compression gripping member defining a gripping member bore to receive the interventional device therethrough; and a loading mechanism operable to deform the compression gripping member to grip the interventional device extending through the gripping member bore.

### RELATED APPLICATION(S)

The present application claims the benefit of and priority from U.S. Provisional Patent Application No. 62/128,623, filed Mar. 5, 2015, the disclosure of which is incorporated herein by reference in its entirety.

### FIELD OF THE INVENTION

The present invention relates generally to medical devices and methods and, more particularly, to image-guided devices and methods such as MRI-guided devices and methods.

### BACKGROUND OF THE INVENTION

Trajectory guide frames are employed for image-guided surgeries. Examples of such trajectory guide apparatus are disclosed in U.S. Published Patent Application No. 2009/0112084 A1, the disclosure of which is incorporated herein by reference.

### SUMMARY OF THE INVENTION

According to embodiments of the invention, a trajectory guide frame for use with an image-guided interventional system includes a base, an elongate device guide support, a device guide, and a locking device. The base has a patient access aperture formed therein. The elongate device guide support is secured to the base and has opposite proximal and distal ends. The distal end is positioned proximate the patient access aperture. The device guide support includes a device guide support bore therethrough that extends from the proximal end to the distal end. The device guide is configured to be removably inserted in the device guide support bore. The device guide includes a device guide lumen configured to removably receive an interventional device therethrough. The locking device is configured to secure the interventional device to the device guide and/or the device guide support. The locking device includes a gripping mechanism including: a compression gripping member defining a gripping member bore to receive the interventional device therethrough; and a loading mechanism operable to deform the compression gripping member to grip the interventional device extending through the gripping member bore.

The compression gripping member may be formed of an elastomeric material.

In some embodiments, the loading mechanism is operable to apply an adjustable load to the compression gripping member. In some embodiments, the loading mechanism includes a threaded member that is rotatable to selectively apply and adjust a compressive load on the compression gripping member.

According to some embodiments, the locking device further includes a second gripping mechanism operable to receive and grip the interventional device. In some embodiments, the second gripping mechanism is operable to apply an adjustable gripping force on the interventional device. In some embodiments, the second gripping mechanism includes a threaded member that is rotatable to selectively apply and adjust a compressive gripping load on the interventional device.

According to some embodiments, the device guide support and the locking device each include interlock features to cooperatively releasably secure the locking device to the device guide support.

In some embodiments, the locking device includes a clamping mechanism to releasably secure the locking device to the device guide and/or the device guide support.

In some embodiments, the locking device includes visual reference indicia thereon to assist an operator in axially aligning the interventional device with the locking device.

The trajectory guide frame may include a yoke movably mounted to the base and rotatable about a roll axis, and a platform movably mounted to the yoke and rotatable about a pitch axis, wherein the device guide support is secured to the platform. In some embodiments, the platform includes an X-Y support table movably mounted on the platform to move in an X-direction and a Y-direction substantially perpendicular to the X-direction relative to the platform, and the device guide support is secured to and projects from the X-Y support table.

According to embodiments of the invention, a locking device for securing an interventional device to a device guide and/or a device guide support includes a gripping mechanism. The gripping mechanism includes a compression gripping member and a loading mechanism. The compression gripping member defines a gripping member bore to receive the interventional device therethrough. The loading mechanism is operable to deform the compression gripping member to grip the interventional device extending through the gripping member bore.

The compression gripping member may be formed of an elastomeric material.

According to embodiments of the invention, a method for securing an interventional device to a device guide and/or a device guide support includes providing a locking device including a gripping mechanism including: a compression gripping member defining a gripping member bore to receive the interventional device therethrough; and a loading mechanism operable to deform the compression gripping member to grip the interventional device extending through the gripping member bore. The method further includes: inserting the interventional device into the gripping member bore; and operating the loading mechanism to deform the compression gripping member to grip the interventional device.

In some embodiments, the method further includes removably securing the locking device to the device guide and/or the device guide support.

The compression gripping member may be formed of an elastomeric material.

According to embodiments of the invention, a trajectory guide frame for use with an image-guided interventional system includes a base, a yoke, a platform, an elongate device guide support, and a device guide. The base has a patient access aperture formed therein. The yoke is movably mounted to the base and rotatable about a roll axis. The platform is movably mounted to the yoke and rotatable about a pitch axis. The elongate device guide support is secured to the platform and has opposite proximal and distal ends. The distal end is positioned proximate the patient access aperture. The device guide support includes a device guide support bore therethrough that extends from the proximal end to the distal end. The device guide support includes a proximal end portion projecting from the platform. The proximal end portion extends above the platform by a device guide support height distance in the range of from about 2 cm to 3 cm. The device guide is configured to be removably inserted in the device guide support bore. The device guide includes a device guide lumen configured to removably receive an interventional device therethrough.

In some embodiments, the device guide is a targeting cannula.

In some embodiments, the platform includes an X-Y support table movably mounted on the platform to move in an X-direction and a Y-direction substantially perpendicular to the X-direction relative to the platform, and the device guide support is secured to the X-Y support table, and the device guide support height distance extends from the X-Y support table.

Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a block diagram of an exemplary image-guided interventional system, according to embodiments of the present invention.

FIG. 2 illustrates a user interface that displays and allows a user to adjust the trajectory of a surgical tool such as a targeting cannula, according to embodiments of the present invention.

FIG. 3 illustrates a trajectory guide frame according to embodiments of the present invention secured to the skull of a patient and illustrates a desired trajectory for an interventional device, and also illustrates the actual trajectory of the interventional device as oriented by the frame.

FIG. 4 illustrates the trajectory guide frame of FIG. 3 after reorientation via manipulation of one or more trajectory frame actuators such that the actual trajectory is adjusted to be in alignment with the desired trajectory.

FIG. 5 is an enlarged, fragmentary, top perspective view of the trajectory guide frame of FIG. 3.

FIG. 6 is a partial exploded, top perspective view of the trajectory guide frame of FIG. 3 and a targeting cannula.

FIG. 7 is a cross-sectional view of the trajectory guide frame of FIG. 6 with the targeting cannula installed therein.

FIG. 8 is a partial exploded, top perspective view of the trajectory guide frame of FIG. 3, illustrating a device guide, a device guide adapter, and a depth stop according to embodiments of the invention along with a drill and a drill bit.

FIG. 9 is a cross-sectional, assembled view of the trajectory guide frame of FIG. 8 holding the device guide, the device guide adapter, the depth stop, the drill and the drill bit.

FIG. 10 is a partial exploded, top perspective view of the trajectory guide frame of FIG. 3, the device guide, the depth stop, a locking device, and a biopsy needle according to embodiments of the invention.

FIG. 11 is a cross-sectional assembled view of the trajectory guide frame of FIG. 10 with the device guide, the device guide adapter, the depth stop, the locking device and the biopsy needle.

FIG. 12 is an enlarged, fragmentary view of a portion of FIG. 11.

FIG. 13 is an exploded, top perspective view of the locking device of FIG. 10.

FIG. 14 is an exploded, bottom perspective view of the locking device of FIG. 10.

FIG. 15 is a cross-sectional view of the locking device of FIG. 10 taken along the line 15-15 of FIG. 13.

FIG. 16 is a front view of the locking device of FIG. 10 with the biopsy needle of FIG. 10 mounted therein.

FIG. 17 is a top perspective view of a locking device according to further embodiments of the invention.

FIG. 18 is a cross-sectional view of the locking device of FIG. 17 mounted on a device guide support and a device guide and with a biopsy needle mounted in the locking device.

### DESCRIPTION OF EMBODIMENTS OF THE INVENTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90° or at other orientations) and the spatially relative descriptors used herein interpreted accordingly.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

The term “about”, as used herein with respect to a value or number, means that the value or number can vary by +/−twenty percent (20%).

“Image-guided procedures” and “image-guided surgeries” refer to procedures and surgeries that are executed using imaging system modalities for guidance before and/or during the procedure. In certain embodiments, the imaging modality is a radiation-based imaging modality. Examples of image system modalities include, among other things, magnetic resonance imaging (MRI), computed tomography (CT), positron emission tomography (PET), and single photon emission computed tomography (SPECT) and fluoroscopic systems. In some embodiments, the imaging system includes ultrasound and/or X-ray.

The term “fiducial marker” refers to a marker that can be electronically identified using image recognition and/or electronic interrogation, typically interrogation of CT or MRI image data. The fiducial marker can be provided in any suitable manner, such as, but not limited to, a geometric shape, a component on or in the device, optical or electrical tracking coils, a coating or fluid-filled component or feature (or combinations of different types of fiducial markers) that makes the fiducial marker(s) MRI-visible or CT-visible with sufficient signal intensity (brightness) for identifying location and/or orientation information for the device and/or components thereof in space.

The term “MRI scanner” refers to a magnetic resonance imaging and/or NMR spectroscopy system. As is well known, MRI scanners include a low magnetic field strength magnet (typically between about 0.1 T to about 0.5 T), a medium field strength magnet, or a high-field strength super-conducting magnet, an RF pulse excitation system, and a gradient field system. MRI scanners are well known to those of skill in the art. Examples of commercially available clinical MRI scanners include, for example, those provided by General Electric Medical Systems, Siemens, Philips, Varian, Bruker, Marconi, Hitachi and Toshiba. The MRI systems can be any suitable magnetic field strength, such as, for example, about 1.5 T or about 3.0 T, and may include other high-magnetic field systems between about 2.0 T-10.0 T.

The term “high-magnetic field” refers to field strengths above about 0.5 T (Tesla), typically above 1.0 T, and more typically between about 1.5 T and 10 T.

The term “MRI visible” means that a device is visible, directly or indirectly, in an MRI image. The visibility may be indicated by the increased SNR of the MRI signal proximate to the device (the device can act as an MRI receive antenna to collect signal from local tissue) and/or that the device actually generates MRI signal itself, such as via suitable hydro-based coatings and/or fluid (typically aqueous solutions) filled channels or lumens.

The term “CT visible” means that a device is visible, directly or indirectly, in a CT scan image.

The term “MRI compatible” means that the so-called component(s) is suitable for use in an MRI environment and as such is typically made of a non-ferromagnetic MRI compatible material(s) suitable to reside and/or operate in or proximate a conventional medical high magnetic field environment. The “MRI compatible” component or device is “MR safe” when used in the MRI environment and has been demonstrated to neither significantly affect the quality of the diagnostic information nor have its operations affected by the MR system at the intended use position in an MR system. These components or devices may meet the standards defined by ASTM F2503-05. See, American Society for Testing and Materials (ASTM) International, Designation: F2503-05. Standard Practice for Marking Medical Devices and Other Items for Safety in the Magnetic Resonance Environment. ASTM International, West Conshohocken, Pa., 2005.

The term “near real-time” refers to both low latency and high frame rate. Latency is generally measured as the time from when an event occurs to display of the event (total processing time). For tracking, the frame rate can range from between about 100 fps to the imaging frame rate. In some embodiments, the tracking is updated at the imaging frame rate. For near ‘real-time’ imaging, the frame rate is typically between about 1 fps to about 20 fps, and in some embodiments, between about 3 fps to about 7 fps. The low latency required to be considered “near real time” is generally less than or equal to about 1 second. In some embodiments, the latency for tracking information is about 0.01 s, and typically between about 0.25-0.5 s when interleaved with imaging data. Thus, with respect to tracking, visualizations with the location, orientation and/or configuration of a known intrabody device can be updated with low latency between about 1 fps to about 100 fps. With respect to imaging, visualizations using near real time MR image data can be presented with a low latency, typically within between about 0.01 ms to less than about 1 second, and with a frame rate that is typically between about 1-20 fps. Together, the system can use the tracking signal and image signal data to dynamically present anatomy and one or more intrabody devices in the visualization in near real-time. In some embodiments, the tracking signal data is obtained and the associated spatial coordinates are determined while the MR image data is obtained and the resultant visualization(s) with the intrabody device (e.g., surgical cannula) and the near RT MR image(s) are generated.

The term “targeting cannula” refers to an elongate device, typically having a substantially tubular body that can be oriented to provide positional data relevant to a target treatment site and/or define a desired access path orientation or trajectory. At least portions of a targeting cannula contemplated by embodiments of the invention can be configured to be visible in an MRI image, thereby allowing a clinician to visualize the location and orientation of the targeting cannula in vivo relative to fiducial and/or internal tissue landscape features. Thus, the term “cannula” refers to an elongate device that can be associated with a trajectory frame that attaches to a patient, but does not necessarily enter the body of a patient.

The term “imaging coils” refers to a device that is configured to operate as an MRI receive antenna. The term “coil” with respect to imaging coils is not limited to a coil shape but is used generically to refer to MRI antenna configurations, loopless, looped, etc., as are known to those of skill in the art. The term “fluid-filled” means that the component includes an amount of the fluid but does not require that the fluid totally, or even substantially, fill the component or a space associated with the component. The fluid may be an aqueous solution, MR contrast agent, or any material that generates MRI signal.

The term “tool” refers to devices that facilitate medical procedures.

Embodiments of the invention are particularly suitable for veterinarian or human therapeutic or diagnostic use, but may be used for research or other purposes.

Embodiments of the present invention can be configured to carry out or facilitate image-guided procedures, in particular CT-guided or MRI-guided procedures. Such procedures may include, for example, diagnostic and interventional procedures such as to guide and/or place interventional devices to any desired internal region of the body or object, including deep brain sites for neurosurgeries or other target intrabody locations for other procedures. The object can be any object, and may be particularly suitable for animal and/or human subjects. For example, the system and/or devices thereof can be used for gene, e.g., antibody, and/or stem-cell based therapy delivery or other therapy delivery to intrabody targets in the brain, heart, lungs, liver, kidney, ovary, stomach, intestine, colon, spine or to other locations. In addition, embodiments of the systems can be used to treat cancer sites. In some embodiments, the systems can be used to ablate tissue and/or delivery pharmacologic material in the brain, heart or other locations. In some embodiments, it is contemplated that the systems can be configured to treat AFIB, deliver stem cells or other cardio-rebuilding cells or products into cardiac tissue, such as a heart wall, via a minimally invasive MRI-guided procedure while the heart is beating (i.e., not requiring a non-beating heart with the patient on a heart-lung machine).

Embodiments of the invention are directed to surgical devices that can provide external structural support for intrabody surgical devices. The devices may be configured for CT or MRI environments or may be configured to be compatible for both CT and MRI environments.

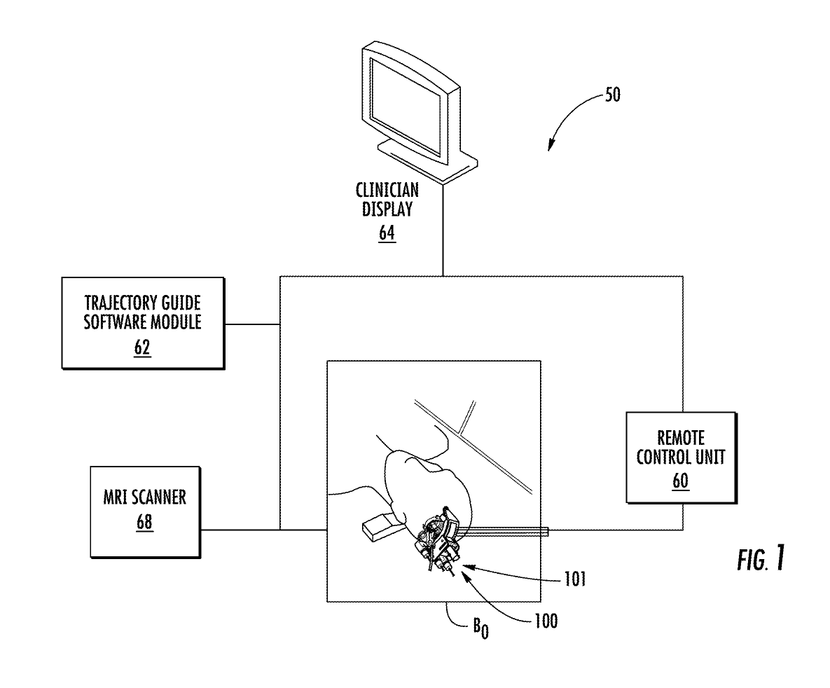

Embodiments of the present invention will now be described in detail below with reference to the figures. FIG. 1 is a block diagram of an image-guided (e.g., MRI-guided) interventional/surgical system 50, according to some embodiments of the present invention. The illustrated system 50 includes an MRI scanner 68, a trajectory guide frame 100 which may be attached to the body of a patient positioned within a magnetic field B₀ of the MRI scanner 68, a remote control unit 60, a trajectory guide software module 62, and a clinician display 64.

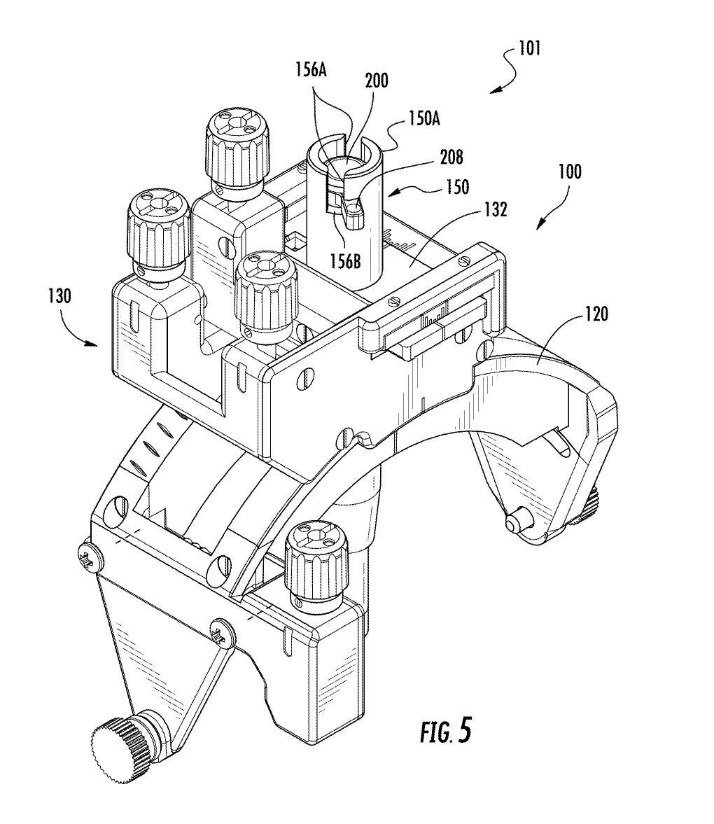

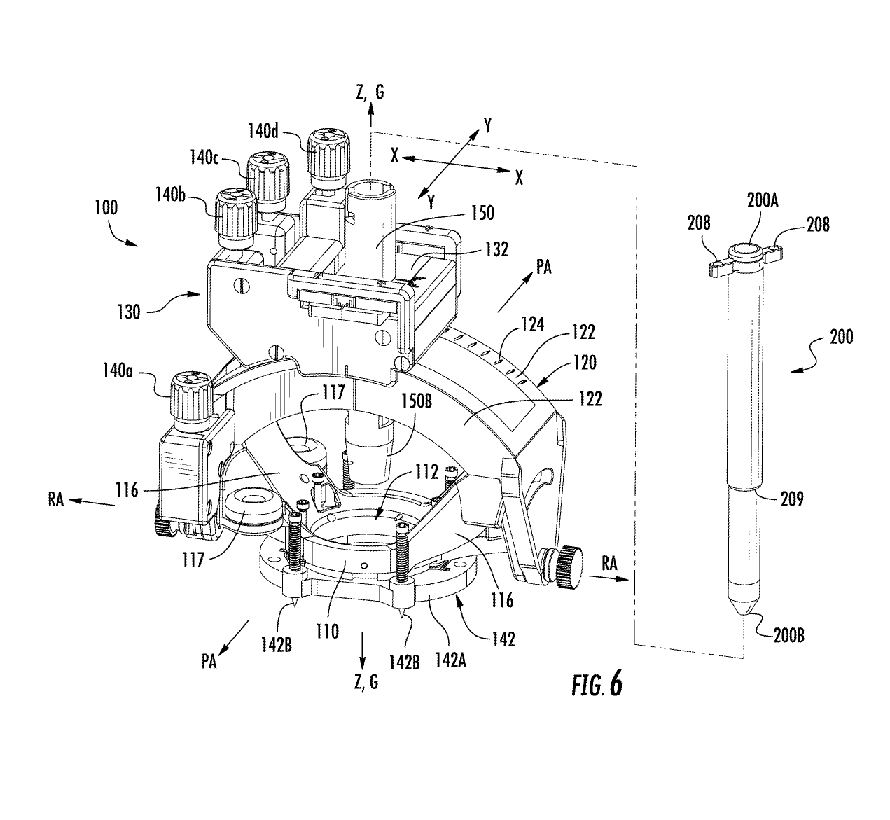

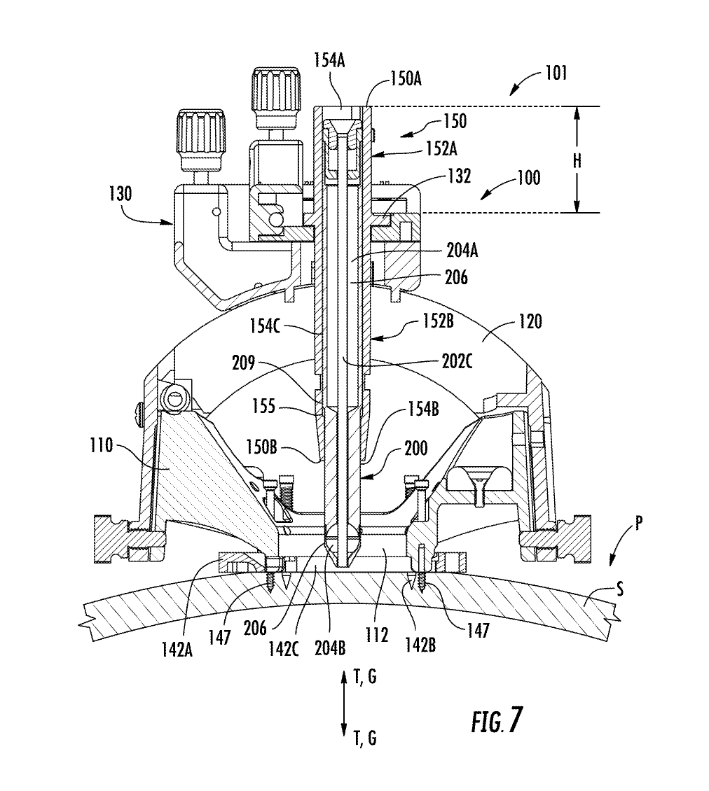

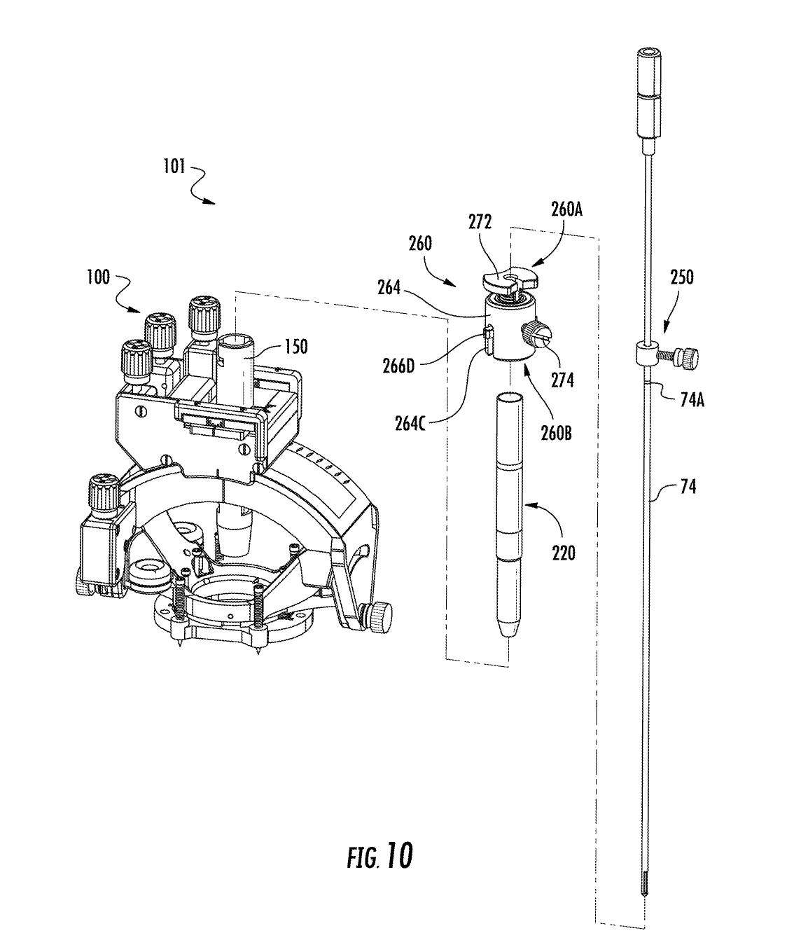

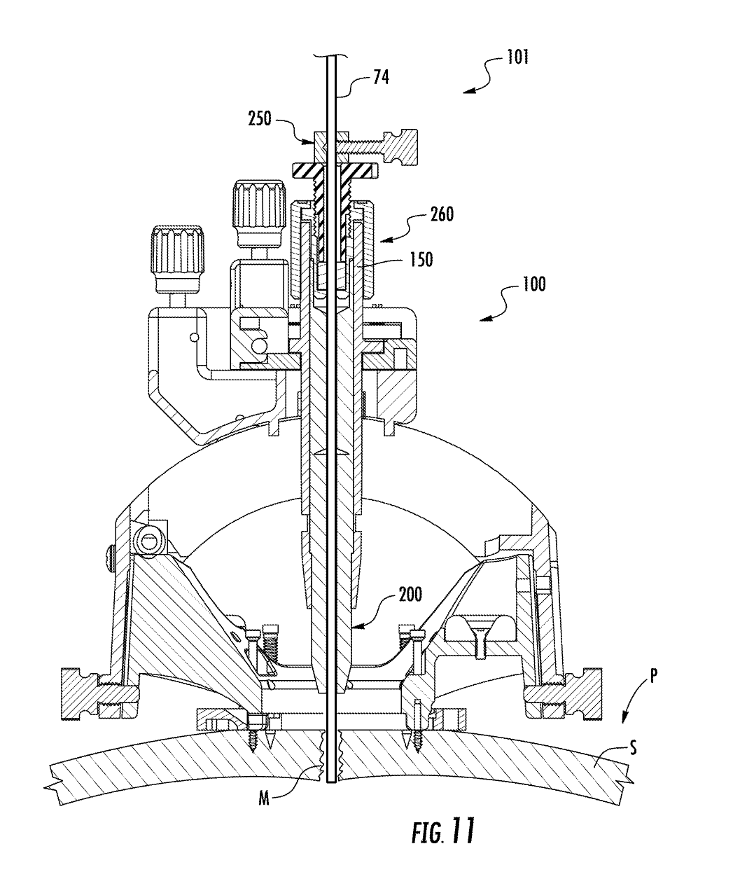

With reference to FIGS. 5-12, a trajectory guide frame system 101 according to embodiments of the present invention is shown therein. The trajectory guide frame system 101 includes the trajectory guide frame 100, a mounting device 142 (FIG. 6), a targeting cannula 200, a device guide 220, a device guide spacer 240, a depth stop 250, a locking device 260 (FIG. 10), and one or more tools or interventional devices 72, 74. Generally and as discussed in more detail herein, the trajectory guide frame 100 is configured to serially support various interchangeable devices including the targeting cannula 200 and the device guide 220 through which various interventional devices may be inserted into the body of a patient. The frame 100 is adjustable such that the targeting cannula 200 and/or device guide 220 is rotatable about a pitch axis, about a roll axis, and the targeting cannula can translate in X-Y directions relative to a Z-direction defined by a device guide support 150 configured to support devices such as the targeting cannula 200 and device guide 220. The frame 100 may be attached to the body of a patient directly or indirectly and may be configured to be attached to various parts of the body.

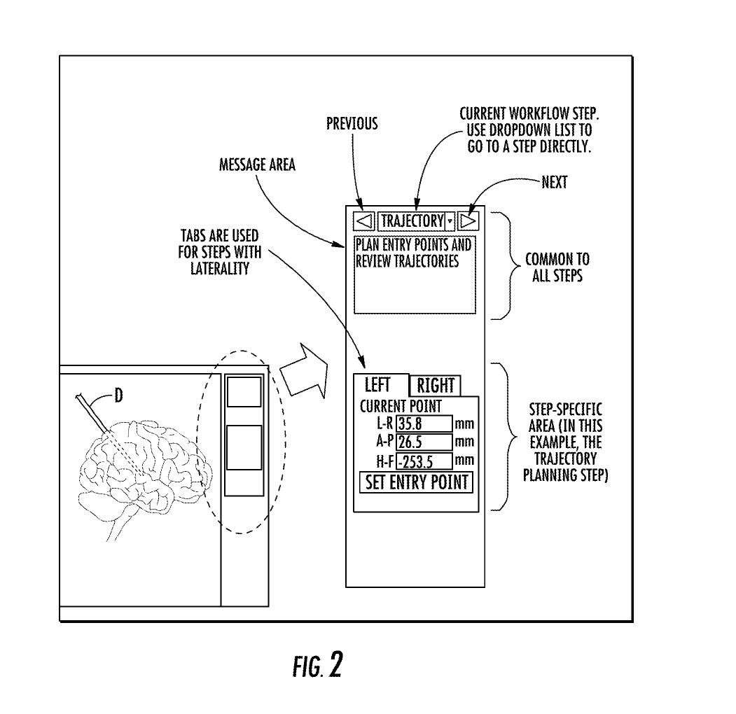

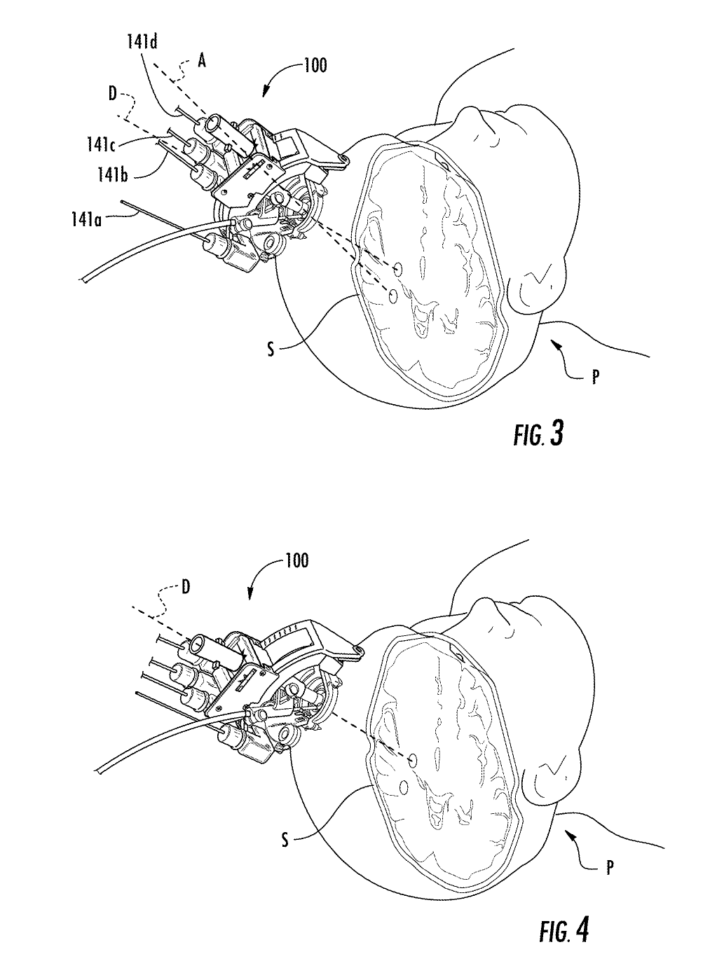

The remote control unit 60 (FIG. 1) allows a user to remotely adjust the position of the targeting cannula 200 or other devices supported by the trajectory guide frame 100. The trajectory guide software module 62 enables a user to define and visualize, via display 64, a desired trajectory (D, FIGS. 2-4) into the body of a patient of an interventional device. The trajectory guide software module 62 also allows a user to visualize and display, via display 64, an actual trajectory (A, FIG. 3) into the body of an interventional device extending through the targeting cannula. The trajectory guide software module 62 displays to the user positional adjustments (FIG. 2) (e.g., pitch axis rotation, roll axis rotation, X-Y translation) that can be used to align the actual trajectory (A) of the targeting cannula with the desired trajectory path (D). In addition, the user can view, via display 64, the actual trajectory changing as he/she adjusts the position of the targeting cannula 200. The trajectory guide software module 62 can be configured to indicate and display when an actual trajectory is aligned with a desired trajectory. The trajectory guide frame 100 may include fiducial markers 117 (FIG. 6) that can be detected in an MRI to facilitate registration of position of the trajectory guide frame 100 in an image. In some embodiments, the image-guided surgical system 50 dynamically renders (in some embodiments, in near real time) the aforementioned displayed components.

The trajectory guide frame 100 allows for the adjustability (typically at least two degrees of freedom, including rotational and translational) and/or calibration/fixation of the trajectory of the targeting cannula 200 or device guide 220 and/or an interventional tool inserted through the targeting cannula 200 or device guide 220.

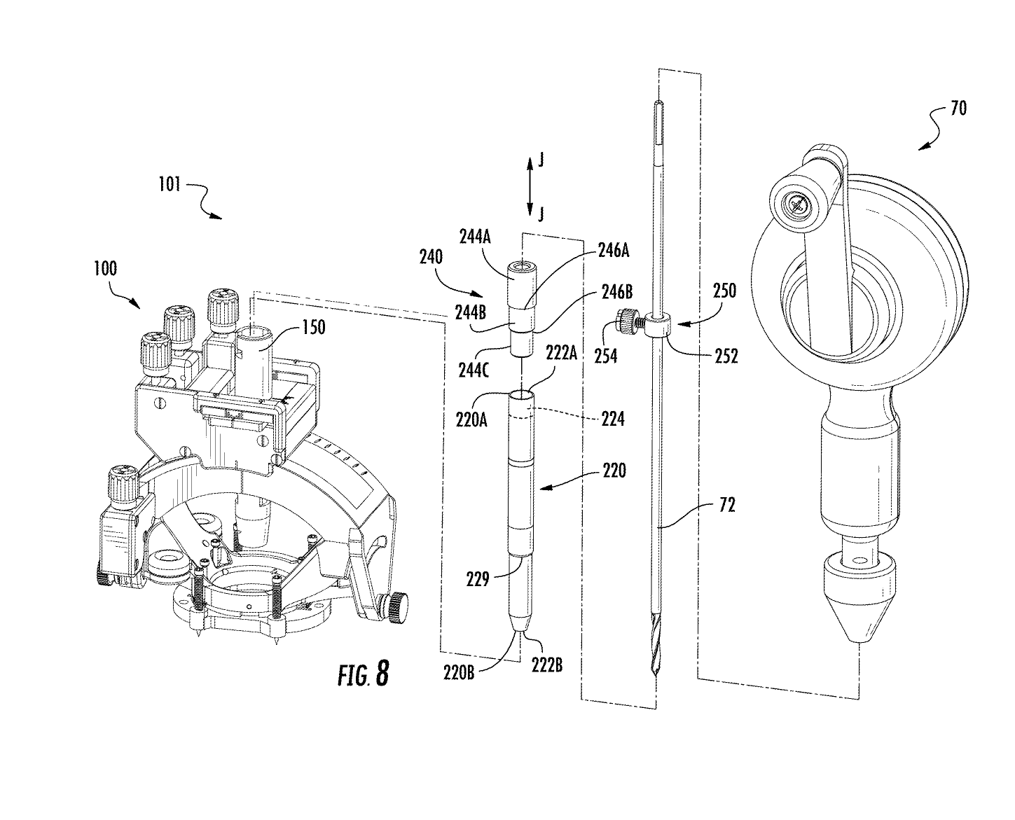

The trajectory guide frame 100 as shown in FIGS. 6 and 7, for example, can include a base 110, a yoke 120, a platform 130, a tubular device guide support 150, and a plurality of actuators 140a-140d. The device guide support 150 is configured to removably receive the targeting cannula 200 and the device guide 220, as described in more detail below.

The base 110 has a patient access aperture 112 formed therein. The base 110 is configured to be secured (directly or indirectly) to the skull of a patient such that the patient access aperture 112 overlies an intended entry location in the patient skull.

Still referring to FIGS. 6 and 7, the platform 130 includes an X-Y support table 132 that is movably mounted to the platform 130. The X-Y support table 132 is configured to move or translate in an X-direction and Y-direction relative to the platform 130 and relative to a Z-direction defined by the longitudinal axis of the device guide support 150. The device guide support 150 is secured to the X-Y support table 132. The device guide support 150 defines a Z-axis along its longitudinal guide support axis G-G relative to the X-Y plane of the X-Y support table 132.

The platform 130 is movably mounted on the yoke 120 to rotate about a pitch axis PA-PA (FIG. 6). The yoke 120 is in turn be movably mounted on the base portion 110 to rotate or pivot about a roll axis RA-RA transverse (in some embodiments, perpendicular) to the pitch axis PA-PA. Thus, the device guide support 150 can be relocated and reoriented by selectively rotating the platform 130 about the pitch axis PA-PA, selectively rotating the yoke 120 about the roll axis RA-RA, and/or selectively translating the support table 132 along its X-axis and/or its Y-axis relative to the yoke 120.

In some embodiments, as shown in FIG. 6, a roll actuator 140a is operably connected to the yoke 120 and is configured to rotate the yoke 120 about the roll axis RA-RA. In some embodiments, the yoke 120 has a range of motion about the roll axis RA-RA of about seventy degrees (70°). However, other ranges, greater and lesser than 70°, are possible, e.g., any suitable angle typically between about 10°-90°, 30°-90°, etc. A pitch actuator 140b is operably connected to the platform 130 and is configured to rotate the platform 130 about the pitch axis PA-PA. In some embodiments, the platform 130 has a range of motion about the pitch axis PA-PA of about seventy degrees (70°). However, other ranges, greater and lesser than 70°, are possible, e.g., any suitable angle typically between about 10°-90°, 30°-90°, etc. An X-direction actuator 140c is operably connected to the platform 130 and is configured to move the X-Y support table 132 in the X-direction. A Y-direction actuator 140d is operably connected to the platform 130 and is configured to move the X-Y support table 132 in the Y-direction.

The actuators 140a-140d are configured to translate and/or rotate portions of the trajectory guide frame 100. The targeting cannula 200 is configured to translate in response to translational movement of the X-Y support table 132 and to rotate in response to rotational movement of the yoke 120 and platform 130 to define different axial intrabody trajectories extending through the patient access aperture 112 in the frame base 110.

The actuators 140a-140d may be manually-operated devices, such as thumbscrews, in some embodiments. The thumbscrews can be mounted on the trajectory guide frame 100 or may reside remotely from the frame 100. A user may turn the actuators 140a-140d by hand to adjust the position of the frame 100 and, thereby, a trajectory of the axis G-G of the device guide support 150. In other embodiments, the actuators 140a-140d are operably connected to a remote control unit 60, for example via a respective plurality of non-ferromagnetic, flexible drive shafts or control cables 141a-141d as described in U.S. Pat. No. 8,374,677 to Piferi et al.

The base 110 also includes a pair of spaced apart arcuate arms 116. The yoke 120 is pivotally attached to pivot points 113 for rotation about the roll axis RA-RA. The yoke 120 engages and moves along the base arcuate arms 116 when rotated about the roll axis RA-RA. In the illustrated embodiment, one of the base arcuate arms 116 includes a thread pattern formed in (e.g., embossed within, machined within, etc.) a surface thereof. However, in other embodiments, both arms 116 may include respective thread patterns. The roll actuator 140a may include a rotatable worm with teeth that are configured to engage the thread pattern. As the worm is rotated, the teeth travel along the thread pattern in the arcuate arm surface. Because the base 110 is fixed to a patient's skull, rotation of the roll actuator worm causes the yoke 120 to rotate about the roll axis RA-RA relative to the fixed base 110.

The yoke 120 includes a pair of spaced apart, upwardly extending, arcuate arms 122. The platform 130 engages and moves along the yoke arcuate arms 122 when rotated about the pitch axis PA-PA. In the illustrated embodiment, one of the yoke arcuate arms 122 includes a thread pattern 124 formed in (e.g., embossed within, machined within, etc.) a surface thereof. However, in other embodiments, both arms 122 may include respective thread patterns. The pitch actuator 140b includes a rotatable worm with teeth that are configured to engage the thread pattern 124. As the worm is rotated, the teeth travel along the thread pattern 124 in the arcuate arm surface. Because the base 110 is fixed to a patient's skull, rotation of the pitch actuator worm causes the platform 130 to rotate about the pitch axis PA-PA relative to the fixed base 110.

The base 110 also includes MRI-visible fiducial markers 117 that allow the location/orientation of the trajectory guide frame 100 to be determined within an MRI image during an MRI-guided procedure. In the illustrated embodiment, the fiducial markers 117 have a torus or “doughnut” shape and are spaced apart. However, fiducial markers having various shapes and positioned at various locations on the trajectory guide frame 100 may be utilized. The fiducial markers 117 may be used to track, monitor, and control the position of the targeting cannula 200 using MRI guidance as described in U.S. Patent Publication No. 2009/0112084 A1, for example.

The mounting device 142 is secured to the bottom of the base 110 (e.g., by fasteners, adhesive or integral formation). The mounting device 142 includes a mounting member 142A and stabilizer or patient engagement structures 142B (hereinafter “pins 142B”), which may take the form of mounting posts, spacers, or pins. The mounting member 142A includes an annular body defining a receiver or access opening 142C aligned with the access aperture 112. The mounting member 142A may be formed of any suitable material that may optionally be MRI-compatible and/or MRI safe material, such as any non-ferromagnetic material and is typically a substantially rigid polymeric material (e.g., polycarbonate). According to some embodiments, the tips of the pins 142B are capable of piercing and penetrating through a scalp upon application of a pressing load by hand. The pins 142B may be formed of any suitable MRI-compatible and/or MRI safe material, such as machined titanium, for example. The mounting device 142 may be constructed and used as described in U.S. Patent Publication No. 2014/0066750 (incorporated herein by reference), for example.

As shown in FIGS. 6-12, the device guide support 150 is elongate and extends from a proximal end 150A to a distal end 150B. The device guide support 150 is attached to the support table 132 at its midsectiOn such that a proximal section 152A of the device guide support 150 extends from the support table 132 to the proximal end 150A and a distal section 152B of the device guide support 150 extends from the support table 132 to the distal end 150B.

In some embodiments, the proximal section 152A extends or projects a height distance II (FIG. 7) above the support table 132. In some embodiments, the height distance H is in the range of from about 2 cm to 3 cm and, in some embodiments, is in the range of from about 2.4 cm to 2.6 cm. The relatively short height distance H reduces the overall height of the trajectory guide frame 100 above the patient, which may be beneficial when the frame 100 is used in a scanner bore or other environment where surgical space is limited.

An axially extending guide bore or passage 154C extends through the device guide support 150 from a proximal or inlet opening 154A to a distal or outlet opening 154B. The bore 154C defines the guide device support axis G-G. An inner circumferentially extending ledge 155 is defined in the bore 154C as shown in FIG. 7.

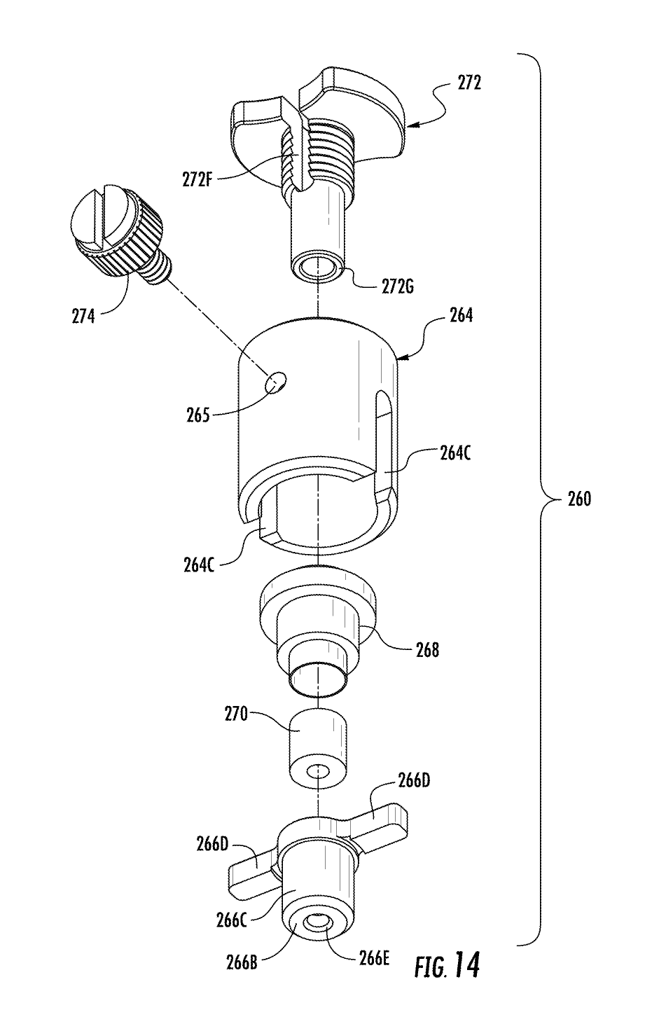

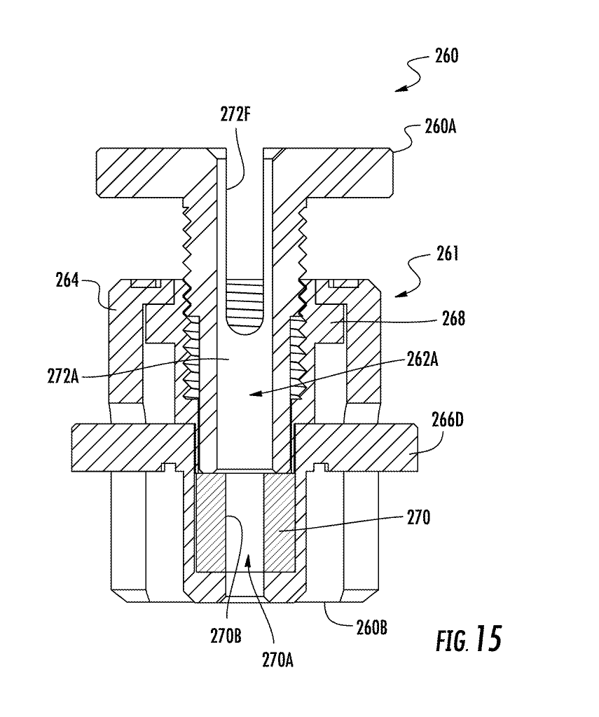

The proximal section 152A includes a pair of opposed, downwardly (axially) extending slots 156A formed therein. As shown in FIGS. 5 and 8, each slot 156A includes a circumferentially extending ledge portion or slot 156B that is configured to engage targeting cannula lugs 208 (FIG. 5) and locking device lugs 266D (FIG. 14) to serially secure the targeting cannula 200 or guide device 220 at a prescribed axial position in the device guide support 150.

The targeting cannula 200 (FIGS. 5-7) is elongate and extends from a proximal end 200A to a distal end 200B. An axially extending guide bore or lumen 202C (FIG. 7) extends through the targeting cannula 200 from a proximal or inlet opening 202A to a distal or outlet opening 202B. The lumen 202C defines a targeting cannula axis T-T (FIG. 7). An outer circumferentially extending ledge 209 is defined about the midsection of the targeting cannula 200.

As shown in FIG. 6, lugs 208 extend outwardly from the proximal end 200A of the targeting cannula 200.

The targeting cannula 200 can include MRI-visible fiducial materials or markers. For example, as illustrated, the targeting cannula 200 may include tubular upper and lower cavities 204A, 204B (which may be fluidly connected) filled with a fiducial material 206 such as an MRI-visible liquid. The fiducial materials or markers may be used to track, monitor, and control the position of the targeting cannula 200 using MRI guidance as described in U.S. Patent Publication No. 2009/0112084 A1, for example.

The lumen 202C of the targeting cannula 200 may be configured to removably receive one or more tools, as described below.

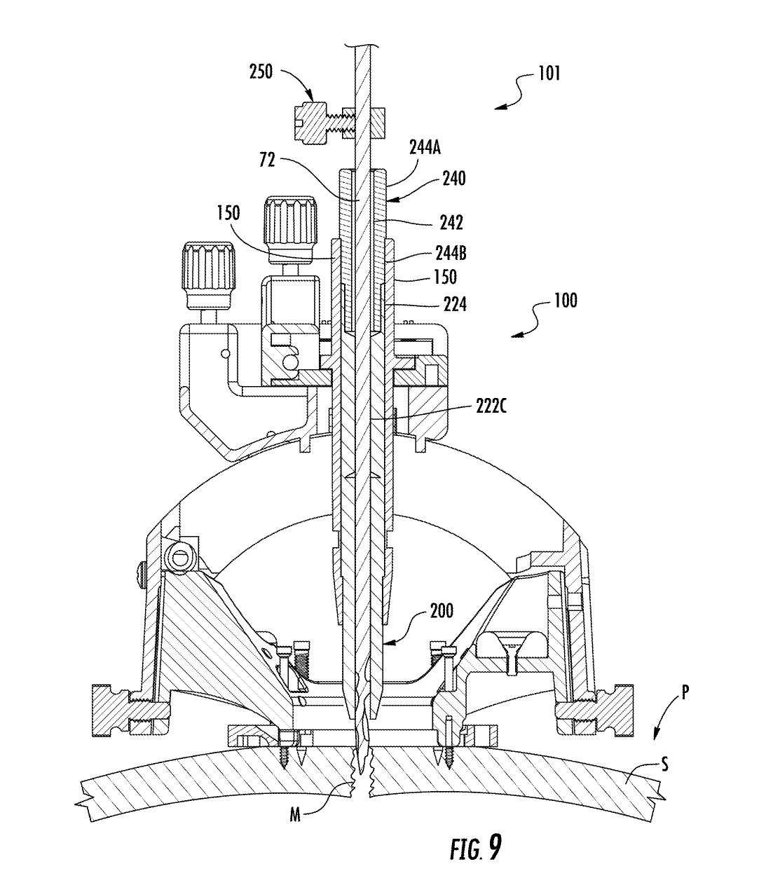

The device guide 220 (FIGS. 8-12) is elongate and extends from a proximal end 220A to a distal end 220B. An axially extending guide bore or lumen 222C extends through the device guide 220 from a proximal or inlet opening 222A to a distal or outlet opening 222B. The lumen 222C defines a device guide axis J-J (FIG. 9). An outer circumferential ledge 229 (FIG. 8) is defined about the midsection of the device guide 220. An upper connection socket 224 is defined in the proximal end of the lumen 222C.

The lumen 222C of the device guide 220 may be configured to removably receive one or more tools, as described below. In some embodiments, lumen 222C of the device guide 220 may have a larger diameter than the lumen 202C of the targeting cannula 200, which thereby allows for various sized devices to be utilized with the frame 100 that otherwise would not be able to do so.

The spacer 240 (FIGS. 8 and 9) is tubular and includes a through bore 242. The spacer 240 includes a proximal section 244A, a midsection 244B and a distal section 244C. The sections 244A, 244B and 244C have sequentially decreasing outer diameters and define upper and lower circumferential outer ledges 246A and 246B.

In some embodiments, the device guide 220 can cooperate with a depth stop 250. The depth stop 250 includes an annular or tubular collar 252 and a thumbscrew 254 threadedly received in the sidewall of the collar 252.

The locking device 260 (FIGS. 10-16) has a proximal end 260A and a distal end 260B. A through bore 262A extends axially through the locking device 260 from an inlet at the end 260A to an outlet at the end 260B. The locking device 260 includes a support collar 264, a guide connector 266, an adapter 268, a compression sealing or gripping member 270, a lock cap 272, a clamping mechanism 274 (as illustrated, a thumbscrew), and a visual reference or alignment indicia 276.

The support collar 264 includes a generally tubular sidewall 264A and an integral, annular end wall 264B. An opening 264E is defined in the end wall 264B. A pair of opposed axially extending side slots 264C and a radially extending screw hole 264D are defined in the side wall 264A. A threaded bore 265 extends radially through the sidewall 264A. The thumbscrew 274 is threadedly mounted in the bore 265.

As shown in FIGS. 13 and 14, the adapter 268 is tubular and includes a proximal section 268A, a midsection 268B, and a distal section 268C. An annular outer flange 268D projects outwardly relative to the midsection 268B. A female thread 268E is provided on the inner diameter of the sections 268A, 268B. The adapter 268 is secured to and mated with the support collar 264 such that the section 268A is received in the opening 264E and the flange 268D is disposed in abutment with or closely adjacent the end wall 264B. In some embodiments, the adapter 268 is bonded (e.g., by adhesive) to the support collar 264.

As also shown in FIGS. 13 and 14, the guide connector 266 includes a tubular sidewall 266C and an integral end wall 266B defining a cavity 266A. An opening 266E is defined in the end wall 266B. An annular outer flange 266E surrounds the proximal end of the guide connector 266. A pair of opposed lugs 266D extend laterally outwardly from the proximal end of the guide connector 266. The guide connector 266 is secured to and mated with the adapter 268 such that the distal section 268C is received in the distal end of the guide connector 266. In some embodiments, the adapter 268 is bonded (e.g., by adhesive) to guide connector 266. When assembled as shown in FIG. 10, the lugs 266D extend through the side slots 264C.

The compression gripping member 270 is annular or tubular and has an inner wall surface 270B defining an axial through bore 270A. The compression gripping member 270 is seated in the cavity 266A in abutment with the end wall 266B.

The compression gripping member 270 can be formed of a deformable material. In some embodiments, the compression gripping member 270 is formed of a resilient, pliable, elastically deformable material. In some embodiments, the compression gripping member 270 is formed of a polymeric material. In some embodiments, the compression gripping member 270 is formed of an elastomeric material or elastomer. In some embodiments, the compression gripping member 270 is formed of a rubber. In some embodiments, the compression gripping member 270 is formed of silicone rubber. Other suitable materials for the compression gripping member 270 may include Buna-N, Neoprene, and/or EPDM, for example.

In some embodiments, the compression gripping member 270 is formed of a material having a durometer in the range of from about 30 A to 50 A Shore and, in some embodiments, in the range of from about 55 A to 70 A Shore.

The lock cap 272 includes a proximal section 272B, a midsection 272C, and a distal section 272D. The proximal section 272B may be ergonomically shaped to provide a handle feature that facilitates manipulation by finger or hand, as illustrated. An outer male thread 272E is located on the midsection 272C. A through bore 272A extends axially through the lock cap 272. A viewing slot 272F extends axially and radially through the sections 272B, 272C. The distal section 272B is slidably received in the guide connector 266. The thread 272E is operatively mated with the thread 268E. At assembly, the compression gripping member 270 is captured in the cavity 266A between the distal end face 272G of the lock cap 272 and the guide connector 266 as shown in FIG. 15.

The visual reference indicia (e.g., mark) 276 is located on the top face of the end wall 264B such that it is visible through the viewing slot 272F. The mark 276 may be formed of a visually identifiable feature and/or color such as a rib, or a mark of an ink or an ink and epoxy composition, coating, paint or other feature, for example.

The components of the trajectory guide frame 100, the targeting cannula 200, the spacer 240, the depth stop 250 and the locking device 260 may be formed of any suitable material and, in some embodiments are formed of an MRI-compatible and/or MRI safe material, such as any non-ferromagnetic material. With the exception of the fiducial materials, the compression gripping member 270, and the visual reference mark 276, the components of the trajectory guide frame 100, the targeting cannula 200, the spacer 240, the depth stop 250 and the locking device 260 are typically formed of a substantially rigid polymeric material (e.g., polycarbonate).

According to some method embodiments of the invention, the system 101 and the trajectory guide frame 100 may be used as follows to execute surgical procedures or interventions. However, it will be appreciated these methods may be modified and the systems and frame may be used for other types of procedures. Although the device guide support 150, the targeting cannula 200, the device guide 220, and the locking device 260 are shown for use with a frame system 101, other trajectory guide frames 100 and/or systems 101 may be used.

Initially, a patient is placed within an MR scanner and MR images are obtained of the patient's head that visualize the patient's skull, brain, fiducial markers and ROI (region of interest or target therapeutic site). The MR images can include volumetric high-resolution images of the brain. To identify the target ROI, certain known anatomical landmarks can be used, i.e., reference to the AC, PC and MCP points (brain atlases give the location of different anatomies in the brain with respect to these points) and other anatomical landmarks. The location of the intended patient access hole may optionally be determined manually by placing fiducial markers on the surface of the head or programmatically by projecting the location in an image.

Images in the planned plane of trajectory are obtained to confirm that the trajectory is viable, i.e., that no complications with anatomically sensitive areas should occur. The patient's skull is optically or manually marked in one or more desired locations to drill an access hole.

The trajectory guide frame 100 is then fixed to the skull S of the patient P. For example, the mounting device 142 can be affixed to the patient's skull using screws 147. Alternatively, in some embodiments, the trajectory guide frame 100 can be held above or over the patient anatomy by a supplemental support, for example.

With reference to FIG. 6, the targeting cannula 200 is properly fitted to the trajectory guide frame 100. More particularly, the targeting cannula 200 is slid down into the passage 154C of the guide device support 150 until the ledges 209 and 155 abut. The lugs 208 slide along the slots 156A to allow the targeting cannula 200 to be inserted within the device guide support 150. The targeting cannula 200 is then rotated about the axis G-G to seat and interlock the lugs 208 in the ledge slots 156B. In this manner, the targeting cannula 200 can be securely held at a prescribed axial position, as shown in FIG. 7.

With the targeting cannula 200 installed in the device guide support 150, a localization scan can be obtained to determine/register the location of the targeting cannula 200, in direct orientation of the trajectory guide frame 100. The settings to which the trajectory guide frame 100 should be adjusted are electronically determined so that the targeting cannula 200 is in the desired trajectory plane. Frame adjustment calculations are provided to a clinician who can manually or electronically adjust the orientation of the trajectory guide frame 100. The desired trajectory plane is confirmed by imaging in one or more planes orthogonal to the desired trajectory plane. According to some embodiments, the positioning of the targeting cannula 200 is conducted in an MRI scanner and is MRI-guided.

After the targeting cannula 200 is aligned (i.e., has the desired trajectory plane), a center punch (not shown) can be placed down the targeting cannula lumen 202C and pushed or tapped into the skull of a patient. This will create an incision in the scalp and provide a starting point for a drill bit. Alternately, an incision can be made in a patient's scalp first. In some instances, a center punch may not be required.

With reference to FIGS. 8 and 9, the targeting cannula 200 is removed from the frame 100 and a drill 70 and drill bit 72 are then used to drill an access hole M in the patient's skull. The system 101 may be prepared as follows for the drilling operation. The device guide 220 is inserted into the device guide support 150 until the ledges 229 and 155 abut. The distal section 244C of the spacer 240 is inserted into the device guide socket 224 as shown in FIG. 9. The depth stop 250 is slid onto the drill bit 52 to the desired drilling depth and secured in place using the thumbscrew 254. The drill bit 72 is then inserted through the bore 242 of the spacer 240 and the bore 222C of the device guide 220 such that the lead end of the drill bit 72 projects beyond the distal end 220B and into engagement with the patient's skulls.

The drill bit 72 is then advanced through the bore 154C and the drill 70 is operated to rotatively drive the drill bit 72 to form the access hole M.

Once the access hole M has been drilled in the skulls of the patient using the drill bit 72, the drill bit 72 and the adapter 240 are removed from the device guide support 150. The device guide 220 may also be removed (e.g., if it will not be used in the next step).

Once the access hole M has been formed in the patient's head, a multipurpose probe (not shown) can be advanced through the device guide 220, the targeting cannula 200, or another device guide installed in the passage 154C of the device guide support 150. The advancement of the probe can be monitored by imaging to verify that the probe will reach the target accurately. If the probe is not at the desired/optimal location, the probe is removed and a decision is made as to where the probe needs to be. The trajectory guide frame 100 is adjusted accordingly via the actuators 140a-140d and the probe is re-advanced into the brain. Once the probe is at the desired location, the probe is removed.

Following the drilling step (or the probing step, if executed), the device guide 220 is re-installed in the device guide support passage 154C if removed.

Once the trajectory has been confirmed, an interventional (e.g., surgical, diagnostic, or biopsy) step may be executed using an interventional device 74. The illustrated interventional device 74 is a biopsy needle. However, other types and configurations of interventional devices may be employed, and methods of the present invention are not limited to the use of a biopsy needle. Such other types of interventional devices may include one or more of the following: an infusion cannula, an ablation tool, a shunt catheter, and a sheathed lead with electrodes. Moreover, any number of interventional steps may be executed using the system 101 as described.

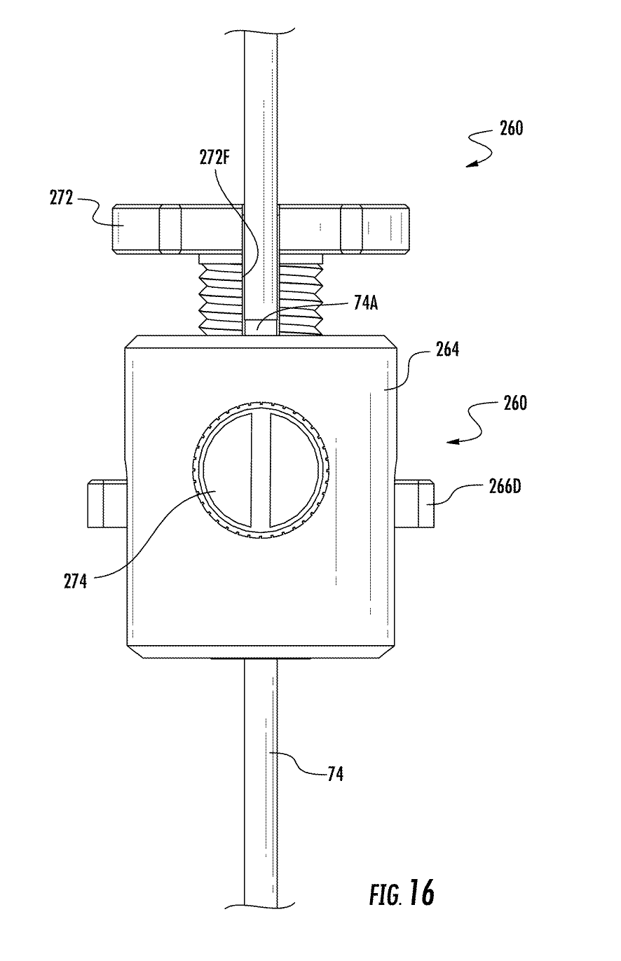

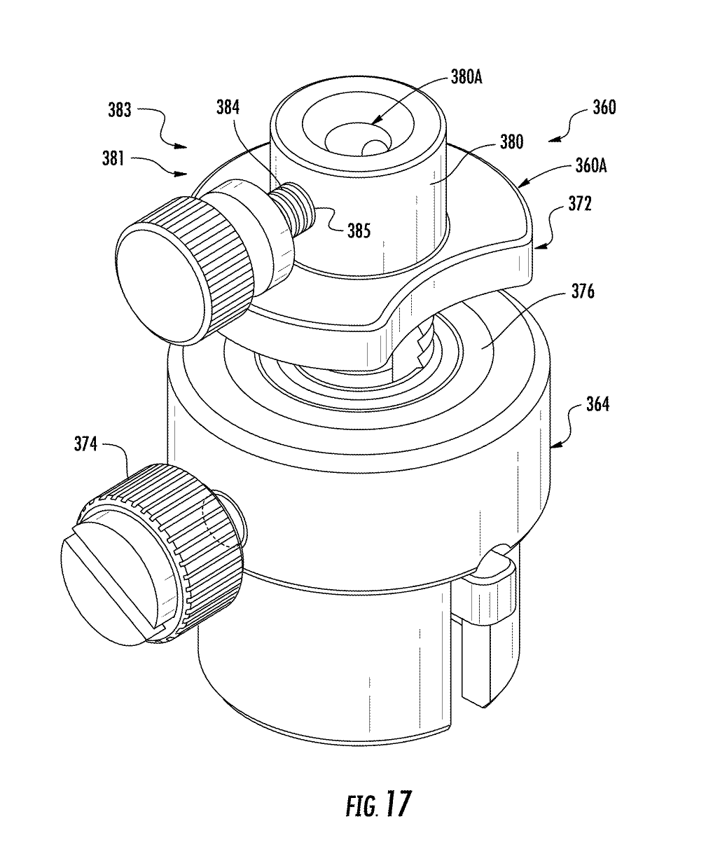

With reference to FIGS. 10-12, depth mark 74A (FIGS. 10 and 16) is made on the needle 74 corresponding to the desired insertion depth for the needle 74 into the patient. The depth stop 250 is temporarily secured to the needle 74 at location above the depth mark 74A.

The lock cap 272 of locking device 260 is set at a first, relatively raised position such that the lock cap 272 does not compress (i.e., load and axially shorten) the compression gripping member 270 or only compresses the compression gripping member 270 to a first extent so that the bore 270A has a first nominal inner diameter.

The needle 74 is then slid into the locking device bore 262C until the depth mark 74A is substantially axially aligned with the visual reference marking 276 (FIG. 13). The position of the depth mark 74A can be observed and monitored through the viewing slot 272F.

With the needle 74 properly positioned, the lock cap 272 is then rotated about the needle 74 and relative to the adapter 268 to screw the lock cap 272 down, reducing the axial spacing between the end 272G and the end wall 266B. The compression gripping member 270 is thereby axially compressively loaded and axially shortened to a second extent greater than the first extent. The compression gripping member 270 is thereby deformed such that the inner wall 270B deforms, protrudes or bulges radially inwardly and reduces the nominal inner diameter of the bore 270A. The lock cap 272 is rotated in this manner until the compression gripping member 270 exerts a sufficient circumferentially extending or distributed, radial load on the needle 74. In this manner, the needle 74 is gripped by the compression gripping member 270 to the locking device 260.

Thus, the lock cap 272, the threads 268E, 272C, and the guide connector 266 cooperatively operate as an adjustable loading mechanism 261 on the compression gripping member 270. The adjustable loading mechanism 261 and the compression gripping member 270 cooperatively form a gripping mechanism 263. Advantageously, the gripping load is substantially uniformly distributed about the circumference of the needle 74 so that the risk of damaging or skewing the needle 74 is reduced. The locking device 260 and the compression gripping member 270 can be particularly suitable to releasably engage fragile, frangible, or breakable tools (such as a borosilicate needle or cannula) and/or can distribute holding forces for better axial position locking.

The threaded engagement between the lock cap 272 and the adapter 268 can provide a continuously adjustable loading mechanism so that the load of the compression gripping member 270 can be selectively adjusted to properly secure interventional devices within a range of diameters.

According to some embodiments, when the locking device 260 is installed on the interventional device 74, the compression gripping member 270 applies a radial load on the interventional device 74 in the range of from about 1 to 3 lbs.

With the locking device 260 secured to the needle 74, the depth stop 250 can be loosened and slid down onto the top of the locking device 260. The depth stop 250 is then retightened onto the needle 74.

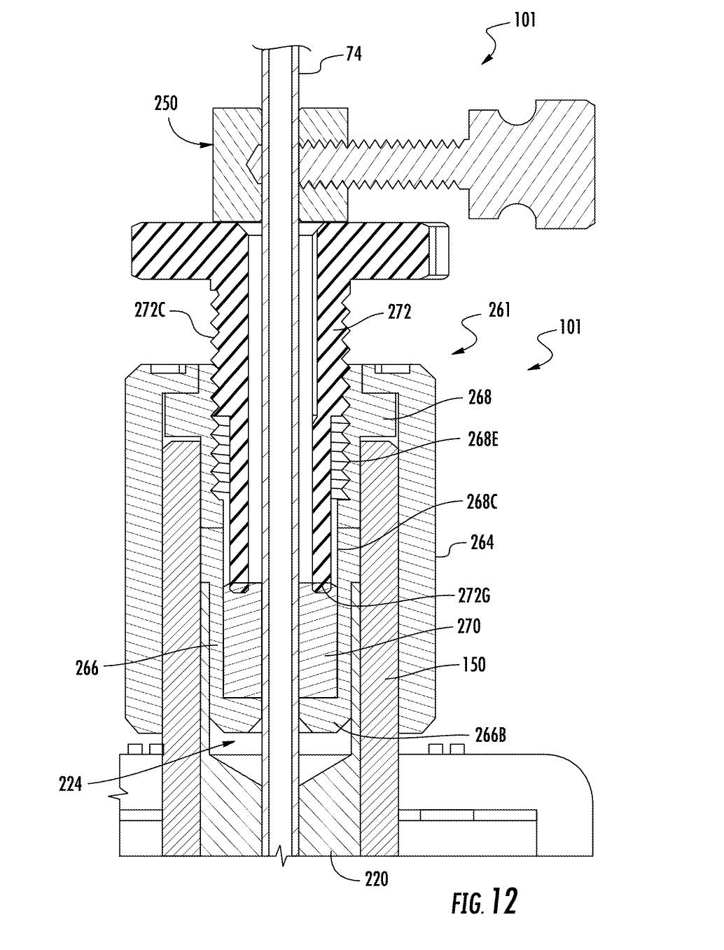

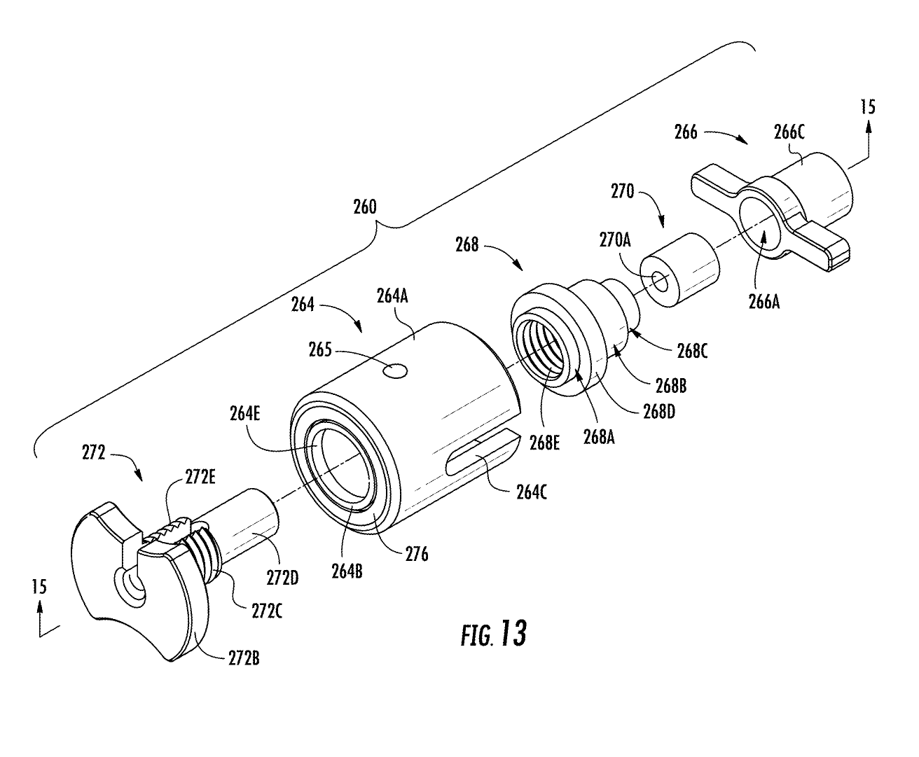

The subassembly of the needle 74, the depth stop 250 and the locking device 260 can be mounted on the device guide support 150. More particularly, the support collar 264 is slid over the device guide support 150 such that the lugs 266D slide down along the slots 156A. The guide connector 266 of the locking device 260 is received into the device guide socket 224 as shown in FIGS. 11 and 12. The needle 74 slides down through the lumen 222C and the hole M in the skull S, and into the patient's brain, for example. The support collar 264 is then rotated about the axis G-G to seat and interlock the lugs 266D in the ledge slots 156B. The thumbscrew 274 is then tightened to clamp the device guide support 150 between the thumbscrew 274 and the adapter 268. In this manner, the locking device 260 and the biopsy needle 74 can be securely held at a prescribed axial position.

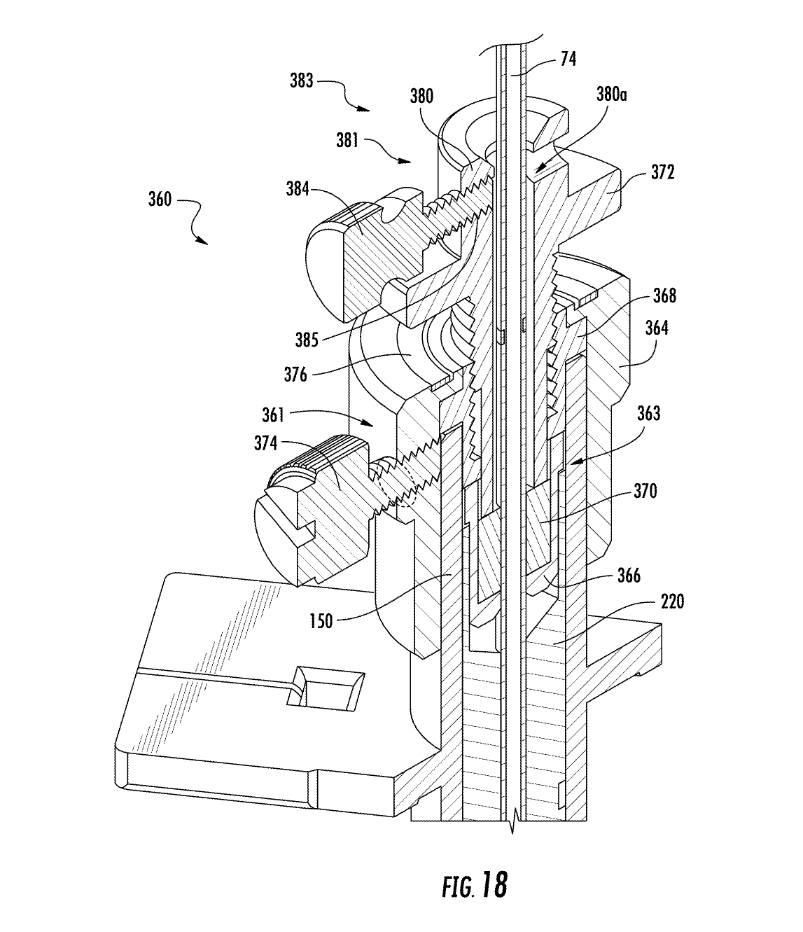

With reference to FIGS. 17 and 18, a locking device 360 according to further embodiments of the invention is shown therein. The locking device 360 corresponds to and may be constructed and used in the same manner as described herein for the locking device 260, except as follows. The locking device 360 includes a support collar 364, a guide connector 366, an adapter 368, a gripping member 370, a lock cap 372, a clamping mechanism (thumbscrew) 374, alignment indicia 376, and an adjustable loading mechanism 361 corresponding to the components 264, 266, 268, 270, 272, 274, 276 and 261 of the locking device 260.

The lock cap 372 of the locking device 360 further includes an upstanding tubular flange 380 extending from the proximal end 360A. The through bore 372A of the lock cap 370 extends through the flange 380. An internally threaded bore 385 extends radially through the flange 380 and intersects the bore 372A. A clamping device in the form of an externally threaded thumbscrew 384 is threadedly mounted in the bore 385. The thumbscrew 384 and the threaded bore 385 cooperatively operate as a second adjustable loading mechanism 381. The second adjustable loading mechanism 381 and the flange 380 cooperative operate as a second gripping mechanism 383.

The locking device 360 can be mounted on an interventional device 74 (e.g., a biopsy needle) as described above using the first gripping mechanism 363 (including the first adjustable loading mechanism 361) such that the device 74 is grasped at a first, lower axial location by the compression gripping member 370. Additionally, the thumbscrew 384 is tightened to bear against and radially load the section of the device 74 in the flange 380 against the flange 380 to grasp the device 74 at a second, upper location. In this manner, the device 74 can be further secured in the locking device 360 using the second adjustable loading mechanism 381 and the second gripping mechanism 383.

The second gripping mechanism 383 can provide additional security and versatility. When the locking device 360 is used to secure a relatively fragile interventional device such as a laser fiber or optical fiber, the first gripping mechanism 363 may be used alone (i.e., the gripping mechanism 383 is not loaded against the interventional device). When a rigid interventional device such as a biopsy needle is mounted in the locking device 360, both of the adjustable loading mechanisms 363 and 383 may be used together.

It is contemplated that embodiments of the invention can provide an integrated image-guided system 50 that may allow the physician to place the interventional device accurately and in short duration of time. In some embodiments, once the trajectory frame is fixed to the skull, the trajectory frame is oriented such that the interventional device advanced using the trajectory frame follows the desired trajectory and reaches the target as planned in preoperative setup imaging plans. As described herein, the system 50 can employ hardware and software components to facilitate an automated or semi-automated operation to carry out this objective.

While only two device guides (i.e., the device guide 220 and the targeting cannula 200) are shown and described above, the frame system 101 may include three or more device guides having guide lumens with different size internal diameters for receiving various devices of different sizes. For example, a device guide may have an internal diameter sized to receive a particular device therein. Another device guide may have a larger or smaller internal diameter also sized to receive a particular device therein. To facilitate replacing one size device guide with another, each device guide may be configured to be removably seated in the device guide support 150.

Although described for use with a head (e.g., for brain surgeries), according to other embodiments, the systems 50, 101 may be used to execute surgical interventions at a selected location on the patient other than the skull.

In some embodiments, an access (burr) hole may be formed (e.g., by drilling) in the skull before or after mounting the frame 100 on the skull. In this case the step of drilling using the drill 70 may be omitted.

Trajectory guide frames as disclosed herein can provide a stable platform for advancing surgical devices, leads, etc., in the brain, as described above. However, a trajectory frame according to embodiments of the present invention can be configured to be mounted to various portions of the body of a patient.

It will be appreciated that aspects of the present invention can be used with or incorporated into trajectory guide frames of other types and configurations.

The trajectory guide frame systems of the present invention can be provided as a sterile kit (typically as single-use disposable hardware) or in other groups or sub-groups or tools or even individually, typically provided in suitable sterile packaging. The tools can also include a marking grid (e.g., as disclosed in U.S. Published Patent Application No. 2009-00177077 and/or U.S. Published Patent Application No. 2009/00171184). Certain components of the kit may be replaced or omitted depending on the desired procedure. Certain components can be provided in duplicate for bilateral procedures.

Trajectory guide frame systems in accordance with embodiments of the invention may be used to guide and/or place diagnostic or interventional devices and/or therapies to any desired internal region of the body or object using MRI and/or in an MRI scanner or MRI interventional suite. The object can be any object, and may be particularly suitable for animal and/or human subjects. In some embodiments, the guide apparatus is used to place implantable DBS leads for brain stimulation, typically deep brain stimulation. In some embodiments, the guide apparatus can be configured to deliver tools or therapies that stimulate a desired region of the sympathetic nerve chain. Other uses inside or outside the brain include stem cell placement, gene therapy or drug delivery for treating physiological conditions. Some embodiments can be used to treat tumors. Some embodiments can be used for RF ablation, laser ablation, cryogenic ablation, etc. In some embodiments, the interventional tools can be configured to facilitate high resolution imaging via intrabody imaging coils (receive antennas), and/or the interventional tools can be configured to stimulate local tissue, which can facilitate confirmation of proper location by generating a physiologic feedback (observed physical reaction or via fMRI).

In some embodiments, the trajectory guide frame system is used for delivering bions, stem cells or other target cells to site-specific regions in the body, such as neurological target and the like. In some embodiments, the guide apparatus is used to introduce stem cells and/or other cardio-rebuilding cells or products into cardiac tissue, such as a heart wall via a minimally invasive MRI-guided procedure, while the heart is beating (i.e., not requiring a non-beating heart with the patient on a heart-lung machine) Examples of known stimulation treatments and/or target body regions are described in U.S. Pat. Nos. 6,708,064; 6,438,423; 6,356,786; 6,526,318; 6,405,079; 6,167,311; 6,539,263; 6,609,030 and 6,050,992, the contents of which are hereby incorporated by reference as if recited in full herein.

Generally stated, some embodiments of the invention are directed to MRI interventional procedures including locally placing interventional tools or therapies in vivo to site-specific regions using an MRI system. The interventional tools can be used to define an MRI-guided trajectory or access path to an in vivo treatment site. Some embodiments of the invention provide interventional tools that can provide positional data regarding location and orientation of a tool in 3-D space with a visual confirmation on an MRI. Embodiments of the invention may provide an integrated system that may allow physicians to place interventional devices/leads and/or therapies accurately and in shorter duration procedures over conventional systems.

In some embodiments, MRI can be used to visualize (and/or locate) a therapeutic region of interest inside the brain or other body locations, and to visualize (and/or locate) an interventional tool or tools that will be used to deliver therapy and/or to place a chronically implanted device that will deliver one or more therapies. Then, using the three-dimensional data produced by the MRI system regarding the location of the therapeutic region of interest and the location of the interventional tool, the system and/or physician can make positional adjustments to the interventional tool so as to align the trajectory of the interventional tool, so that when inserted into the body, the interventional tool will intersect with the therapeutic region of interest. With the interventional tool now aligned with the therapeutic region of interest, an interventional probe can be advanced, such as through an open lumen inside of the interventional tool, so that the interventional probe follows the trajectory of the interventional tool and proceeds to the therapeutic region of interest.

In particular embodiments, using MRI in combination with local or internal imaging coils and/or MRI contrast material that may be contained at least partially in and/or on the interventional probe or sheath, the location of the interventional probe within the therapeutic region of interest can be visualized on a display or image and allow the physician to either confirm that the probe is properly placed for delivery of the therapy (and/or placement of the implantable device that will deliver the therapy) or determine that the probe is in the incorrect or a non-optimal location. Assuming that the interventional probe is in the proper desired location, the therapy can be delivered and/or the interventional probe can be removed and replaced with a permanently implanted therapeutic device at the same location.

In some embodiments, in the event that the physician determines from the MRI image produced by the MRI and the imaging coils, which may optionally be contained in or on the interventional probe, that the interventional probe is not in the proper location, a new therapeutic target region can be determined from the MRI images, and the system can be updated to note the coordinates of the new target region. The interventional probe is typically removed (e.g., from the brain) and the interventional tool can be repositioned so that it is aligned with the new target area. The interventional probe can be reinserted on a trajectory to intersect with the new target region. Although described and illustrated herein with respect to the brain and the insertion of deep brain stimulation leads, it is understood that embodiments of the present invention may be utilized at other portions of the body and for various other types of procedures.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A trajectory guide frame for use with an image-guided interventional system, the trajectory guide frame comprising:
a base having a patient access aperture formed therein;
an elongate device guide support secured to the base and having opposite proximal and distal ends, wherein:
the distal end is positioned proximate the patient access aperture; and
the device guide support includes a device guide support bore therethrough that extends from the proximal end to the distal end;
a device guide configured to be removably inserted in the device guide support bore, wherein the device guide includes a device guide lumen configured to removably receive an interventional device therethrough; and
a locking device configured to secure the interventional device to the device guide and/or the device guide support, wherein the locking device includes a gripping mechanism including:
a compression gripping member defining a gripping member bore to receive the interventional device therethrough; and
a loading mechanism operable to deform the compression gripping member to grip the interventional device extending through the gripping member bore.

2. The trajectory guide frame of claim 1 wherein the compression gripping member is formed of an elastomeric material.

3. The trajectory guide frame of claim 1 wherein the loading mechanism is operable to apply an adjustable load to the compression gripping member.

4. The trajectory guide frame of claim 3 wherein the loading mechanism includes a threaded member that is rotatable to selectively apply and adjust a compressive load on the compression gripping member.

5. The trajectory guide frame of claim 1 wherein the locking device further includes a second gripping mechanism operable to receive and grip the interventional device.

6. The trajectory guide frame of claim 5 wherein the second gripping mechanism is operable to apply an adjustable gripping force on the interventional device.

7. The trajectory guide frame of claim 6 wherein the second gripping mechanism includes a threaded member that is rotatable to selectively apply and adjust a compressive gripping load on the interventional device.

8. The trajectory guide frame of claim 1 wherein the device guide support and the locking device each include interlock features to cooperatively releasably secure the locking device to the device guide support.

9. The trajectory guide frame of claim 1 wherein the locking device includes a clamping mechanism to releasably secure the locking device to the device guide and/or the device guide support.

10. The trajectory guide frame of claim 1 wherein the locking device includes visual reference indicia thereon to assist an operator in axially aligning the interventional device with the locking device.

11. The trajectory guide frame of claim 1 including:
a yoke movably mounted to the base and rotatable about a roll axis; and
a platform movably mounted to the yoke and rotatable about a pitch axis;
wherein the device guide support is secured to the platform.

12. The trajectory guide frame of claim 11 wherein:
the platform includes an X-Y support table movably mounted on the platform to move in an X-direction and a Y-direction substantially perpendicular to the X-direction relative to the platform; and
the device guide support is secured to and projects from the X-Y support table.

13. A locking device for securing an interventional device to a device guide and/or a device guide support, the locking device comprising a gripping mechanism including:
a compression gripping member defining a gripping member bore to receive the interventional device therethrough; and
a loading mechanism operable to deform the compression gripping member to grip the interventional device extending through the gripping member bore.

14. The locking device of claim 13 wherein the compression gripping member is formed of an elastomeric material.

15. A method for securing an interventional device to a device guide and/or a device guide support, the method comprising:
providing a locking device including a gripping mechanism including:
a compression gripping member defining a gripping member bore to receive the interventional device therethrough; and
a loading mechanism operable to deform the compression gripping member to grip the interventional device extending through the gripping member bore;
inserting the interventional device into the gripping member bore; and
operating the loading mechanism to deform the compression gripping member to grip the interventional device.

16. The method of claim 15 further including removably securing the locking device to the device guide and/or the device guide support.

17. The method of claim 15 wherein the compression gripping member is formed of an elastomeric material.

18. A trajectory guide frame for use with an image-guided interventional system, the trajectory guide frame comprising:
a base having a patient access aperture formed therein;
a yoke movably mounted to the base and rotatable about a roll axis;
a platform movably mounted to the yoke and rotatable about a pitch axis; and
an elongate device guide support secured to the platform and having opposite proximal and distal ends, wherein:
the distal end is positioned proximate the patient access aperture;
the device guide support includes a device guide support bore therethrough that extends from the proximal end to the distal end; and
the device guide support includes a proximal end portion projecting from the platform, wherein the proximal end portion extends above the platform by a device guide support height distance in the range of from about 2 cm to 3 cm; and
a device guide configured to be removably inserted in the device guide support bore, wherein the device guide includes a device guide lumen configured to removably receive an interventional device therethrough.

19. The trajectory guide frame of claim 18 wherein the device guide is a targeting cannula.

20. The trajectory guide frame of claim 18 wherein:
the platform includes an X-Y support table movably mounted on the platform to move in an X-direction and a Y-direction substantially perpendicular to the X-direction relative to the platform; and
the device guide support is secured to the X-Y support table, and the device guide support height distance extends from the X-Y support table.
